(19) United States
(12) Patent Application Publication
(10) Pub. No.: US 2014/0256004 A1
(43) Pub. Date: Sep. 11, 2014

(54) Process for the Preparation of an Endothelin Receptor Antagonist

(71) Applicant: CIPLA Limited, Mumbai (IN)

(72) Inventors: Dharmaraj Ramachandra Rao, Thane (W) (IN); Rajendra Narayanrao Kankan, Mumbai (IN); Sanjay Naik, Mumbai (IN); Maruti Ghagare, Thane (W) (IN); Sandip Vasant Chikhalikar, Mumbai (IN)

(73) Assignee: CIPLA LIMITED, Mumbai (IN)

(21) Appl. No.: 14/352,917

(22) PCT Filed: Oct. 19, 2012
(86) PCT No.: PCT/GB2012/000798

§ 371 Date: Apr. 18, 2014

(30) Foreign Application Priority Data
Oct. 19, 2011 (IN) 2943/MUM2011

(51) Int. Cl. C12P 7/62; C12P 17/12; C07C 67/313; C07D 239/34; C07C 69/716

(52) U.S. Cl. CPC C12P 7/62; C07D 239/34; C07C 69/716; C07C 67/313; C12P 17/12
USPC 435/122; 544/318; 560/53; 435/135

(57) ABSTRACT

The present invention relates to a novel process for the preparation of a compound of formula (I) wherein R is a methyl or methoxy group; to certain novel intermediates prepared in such a process and their use.

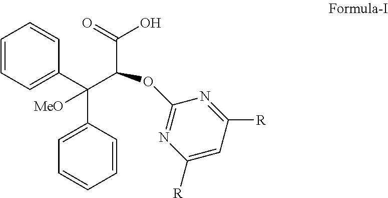

### FIELD OF THE INVENTION

The present invention relates to a novel process for the preparation of a compound of formula (I) and to novel intermediates which are produced during the course of carrying out the novel process:

### BACKGROUND OF THE INVENTION

Compounds of formula (I),

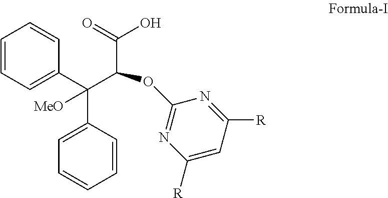

wherein R is a methyl or methoxy group belong to a group of biologically active compounds known as endothelin receptor antagonists.

Endothelins are 21-amino acid vasoconstricting peptides produced primarily in the endothelium having a key role in vascular homeostasis. Endothelin-1 (ET-1) has a number of other actions besides vasoconstriction and cardiac stimulation that can indirectly affect cardiovascular function. Sitaxentan, ambrisentan and bosentan are commercially available endothelin receptor antagonists that are indicated for the treatment of pulmonary arterial hypertension, while atrasentan, also an endothelin receptor antagonist, is an experimental anti-cancer drug.

(S)-2-hydrox-3-methoxy-3,3-diphenylpropionic acid is one of the key intermediate compounds used in the synthesis of endothelin receptor antagonists such as ambrisentan.

Several approaches are described in the literature to make (S)-2-hydroxy-3-methoxy-3,3-diphenylpropionic acid.

U.S. Pat. No. 5,932,730 discloses a process for the preparation of (S)-2-hydroxy-3-methoxy-3,3-diphenyl propionic acid which involves condensation of benzophenone with methyl-2-chloroacetate to obtain racemic 2-hydroxy-3-methoxy-3,3-diphenyl propionic acid, followed by optical resolution with L-proline methyl ester hydrochloride to yield the desired product. In another process variant, there is disclosed resolution of 2-hydroxy-3-methoxy-3,3-diphenyl propionic acid with (S)-1-(nitrophenyl)-ethylamine at a high temperature to obtain 35% of (S)-2-hydroxy-3-methoxy-3,3-diphenyl propionic acid (cf. examples 10 & 11) and further esterified to yield compound of formula (IV). In both of the process variants, the yield is reported to be 35% based on the racemate.

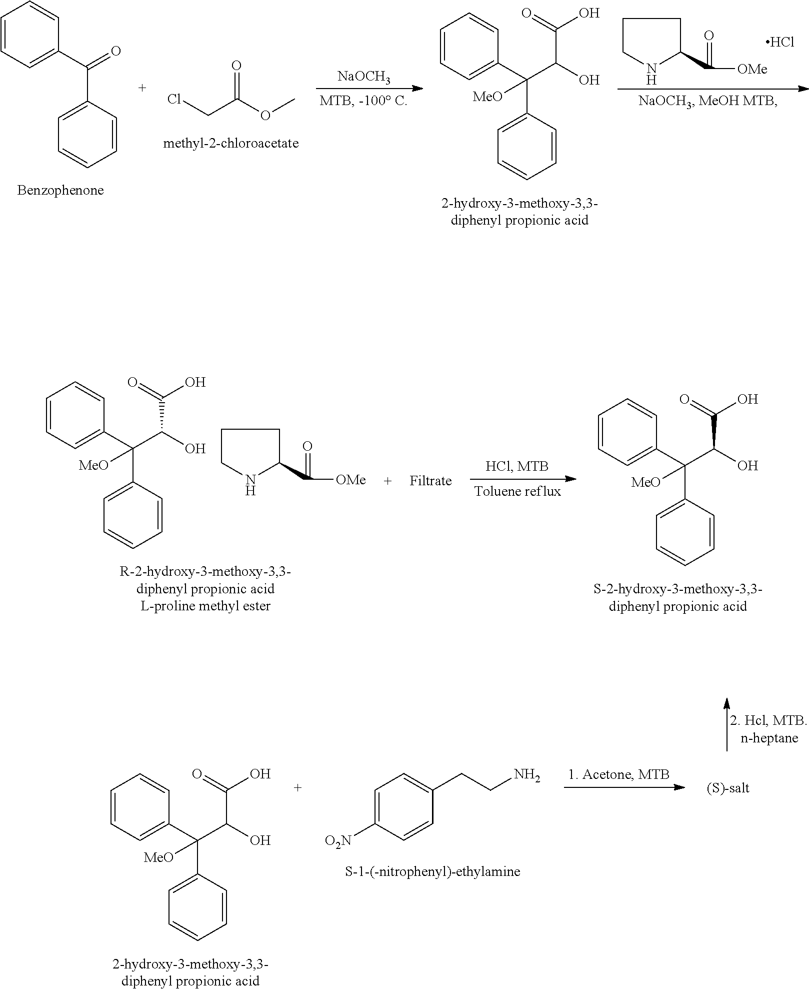

Hence, the processes disclosed in U.S. Pat. No. 5,932,730 require purification of the intermediate compound (S)-2-hydroxy-3-methoxy-3,3-diphenyl propionic acid; otherwise, undesired isomeric impurity carries over to subsequent steps. Moreover, this patent does not disclose conversion of the optically active compound, (S)-2-hydroxy-3-methoxy-3,3-diphenyl propionic acid into an endothelin receptor antagonist of formula (I).

U.S. Pat. No. 6,559,338 describe a process for the chiral resolution of racemic 2-hydroxy-3-methoxy-3,3-diphenylpropionic acid with very expensive optically active (S)-1-(4-chloro phenyl)ethylamine and hence is not suitable for large scale production.

Conversion of the optically active compound (S)-2-hydroxy-3-methoxy-3,3-diphenyl propionic acid into an endothelin receptor antagonist of formula (I) is disclosed in WO2010/091877. The conversion is carried in the presence of the highly toxic and flammable solid, lithium amide. Lithium amide reacts vigorously with water to generate gaseous NH3 and may ignite on contact with water or moist air. Hence the use of lithium amide on a commercial scale is not feasible.

Another process is disclosed in WO2010/091877 wherein, Methyl 3,3-diphenyloxirane-2-carboxylate is converted to Methyl 2-hydroxy-3-methoxy-3,3-diphenylpropanoate, which is hydrolyzed to 2-Hydroxy-3-methoxy-3,3-diphenylpropanoic acid, followed by optical resolution with (S)-1-(4-chlorophenyl)ethylamine and finally isolation of (S)-2-hydroxy-3-methoxy-3,3-diphenyl propionic acid.

A similar process is disclosed in WO2010/070658, wherein 2-Hydroxy-3-methoxy-3,3-diphenylpropanoic acid is optically resolved using L-proline methyl ester hydrochloride, (S)-1-(nitrophenyl)-ethylamine or R(+)-phenyl ethylamine, followed by crystallization to provide (S)-2-hydroxy-3-methoxy-3,3-diphenyl propionic acid.

WO2011004402 describes a process for the chiral resolution of racemic 2-hydroxy-3-methoxy-3,3-diphenyl propionic acid with an optically active chiral amine (with either R or S configuration) to obtain a diastereomeric salt, followed by condensation with 4,6-dimethyl-2-methylsulfonyl pyrimidine to yield ambrisentan. This process involves optical resolution of the intermediate, which leads to about 50% loss of the undesired isomer, multiple steps and hence is time consuming.

The processes disclosed in the prior art are cumbersome. Therefore, there exists a need for a more economical and efficient method of making endothelin receptor antagonists which is suitable for industrial scale-up.

The process of the present invention provides, large scale synthesis of endothelin receptor antagonist having high degree of chromatographic and optical purity and low residual solvent content.

### OBJECTS OF THE INVENTION

The object of the present invention is to provide a novel process for preparing an endothelin receptor antagonist of formula (I).

Yet another object of the present invention is to provide a novel process which proceeds via new chemical intermediates for the synthesis of an endothelin receptor antagonist of formula (I).

Yet another object of the present invention is to provide a process for the synthesis of an endothelin receptor antagonist of formula (I) which is simple, economical and suitable for industrial scale-up.

### STATEMENT OF INVENTION

According to a first aspect of the present invention, there is provided a process for preparing a endothelin receptor antagonist of formula (I),

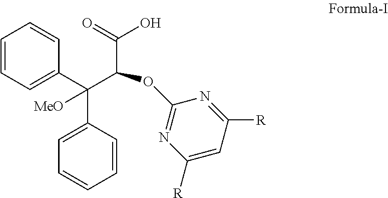

which comprises converting a compound of formula (V)

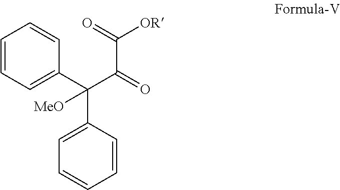

to compound of formula (I), wherein R is a methyl or methoxy group, preferably methyl, and R′ in formula (V) is a lower alkyl group, preferably a straight or branched C₁-C₆ alkyl group such as methyl, ethyl, propyl, butyl, pentyl or hexyl.

In an embodiment, the conversion comprises, reduction of a compound of formula (V) with a suitable chiral reducing agent to provide compound of formula (IV)

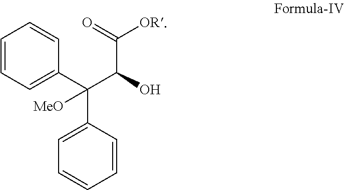

wherein R′ is as defined above.

Preferably, the chiral reducing agent is an enzyme such as a ketoreductase (KRED) or a carbonyl reductase. Most preferably, the chiral reducing agent is a KRED.

In one embodiment, the present invention provides a stereoselective enzymatic reduction process for the preparation of compound of formula (V), a key intermediate in the synthesis of an endothelin receptor antagonist of formula (I), in high enantiomeric purity.

The ketoreductase are capable of reducing the ketone of formula (V), to alcohol of formula (IV), having an enantiomeric purity greater than about 87%, preferably, greater than about 95%, and, more preferably, greater than about 98%, as determined by HPLC.

Compound (IV) as shown above is depicted in the form of the (S)-enantiomer.

Typically, the reduction step is carried in the presence of cofactor for the ketoreductase and optionally a cofactor generating system.

The reduction step is preferably carried out in a co-solvent. The co-solvent assists in enhancing solubility of compounds having poor water solubility, thereby increasing the overall rate of the reaction.

The ratio of water to organic solvent in the co-solvent system is preferably in the range of from about 90:10 to about 95:05 (v/v) water to organic solvent.

The aqueous solvent (water or aqueous co-solvent system) may be pH-buffered or unbuffered. Preferably, the reduction is carried out at a pH of about 10 or below.

During the course of the reduction step, the pH of the reaction mixture may change. The pH of the reaction mixture is preferably maintained at a desired pH or within a desired pH range by the addition of an acid or a base during the course of the reaction. Alternatively, the pH may be controlled by using an aqueous solvent that comprises a suitable buffer.

The reduction step is typically carried out at a temperature in the range of from about −70° C. to about 75° C.

The process of the present invention achieves the stereospecific reduction of ketone to the optically active single isomer of formula (IV). The reduction is highly enantioselective and is therefore advantageous.

Throughout this specification “optically active” is to mean having an enantiomeric excess greater than 97%, preferably greater than 98%, most preferably greater than 99%.

The process of the present invention may further comprise the step of condensing a compound of formula (IV) with a 4,6-disubstituted-2-methyl sulfonyl pyrimidine of formula (III)

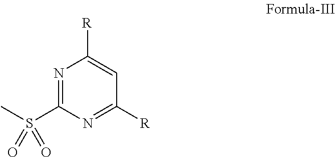

in the presence of a suitable base in a suitable solvent to provide compound of formula (II)

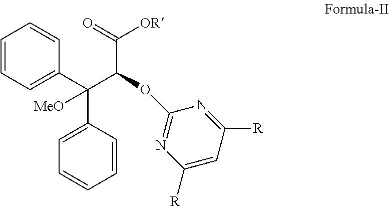

wherein R and R′ are as defined above.

In an embodiment, an optically active single isomer of formula (IV) is condensed with compound of formula (III). The condensation reaction is carried out in the presence of a suitable base in the presence of a suitable solvent.

The condensation step may be carried out at a temperature range of 30° C. to the boiling temperature of the solvent.

In a further embodiment of the invention, the compound of formula (II) may be hydrolyzed in the presence of a suitable base in a suitable solvent to obtain compound of formula (I).

The hydrolysis is typically carried out at a temperature in the range of from about −30° C. to about 50° C.

The endothelin receptor antagonists of formula (I) thereby formed may be optionally purified in asuitable solvent.

Compounds (I), (II) and (IV) as shown above are depicted in the form of the (S)-isomer and are substantially free from (R)-isomer.

The endothelin receptor antagonists of formula (I) are, preferably substantially free from the R -isomer.

In another aspect, the present invention provides a compound of formula (V)

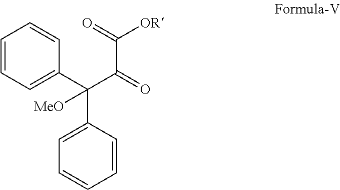

wherein R′ is a lower alkyl group, preferably a straight or branched C₁-C₆ alkyl group, such as methyl, ethyl, propyl, butyl, pentyl or hexyl, preferably methyl, propyl, butyl, pentyl or hexyl, most preferably, methyl.

According to yet another aspect of the present invention, there is provided a process for preparing compound of formula (V), comprising converting a ⁻compound of formula (VI)

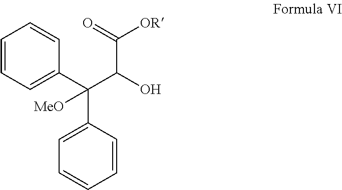

to compound of formula (V), wherein R′ is as defined above.

In an embodiment, the conversion comprises oxidation of compound of formula (VI) using suitable oxidizing agent to yield compound of formula (V).

The process of the present invention is advantageous, as the process for preparing optically active intermediate (IV) does not involve the use of resolving agent for the removal of the undesired isomer, leading to subsequent 50% loss in the yield. The process of the present invention for preparing a compound of formula (IV) reduces or substantially eliminates undesired isomeric impurity to <0.5%. The reaction is carried out at low temperature and is selective, thus it results in formation of optically active intermediate (IV) having enantiomeric purity greater than about 99%.

The present invention also provides compound of formula (V), prepared according to the process described above. Further, the present invention includes optically active compound of formula (V), that is substantially pure and free from other process related impurities and optical impurities.

Further, the present invention provides an endothelin receptor antagonist of formula (I), prepared according to the process described above, having a purity of more than about 99% and a chiral purity of more than about 99% by HPLC.

The endothelin receptor antagonist of formula (I) prepared according to the process of the present invention may be formulated with one or more pharmaceutically acceptable excipients to provide a pharmaceutical composition. Such excipients and compositions are well known to those skilled in the art.

### DETAILED DESCRIPTION OF THE INVENTION

In an embodiment of the present invention, there is provided an improved synthesis of an endothelin receptor antagonist of formula (I), as depicted below in reaction scheme 1.

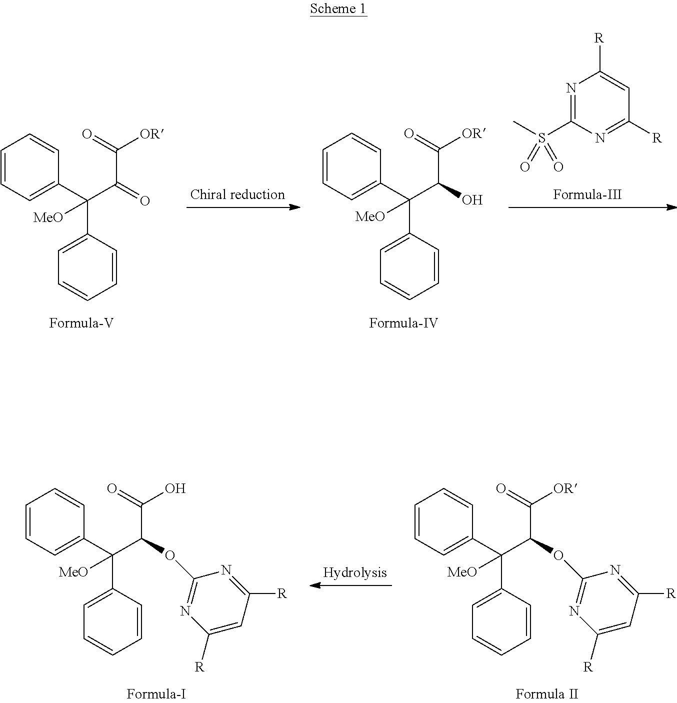

wherein R is a methyl or methoxy group and R′ is an lower alkyl group which may be a straight or branched C₁-C₆ alkyl group, such as methyl, ethyl, propyl, butyl, pentyl or hexyl.

In one embodiment of the invention, the compound of formula (V) is preferably reduced to a compound of formula (IV) using a chiral reducing agent, such as a reducing enzyme, preferably a ketoreductase (KRED) or a carbonyl reductase. Preferably, the chiral reducing agent is a KRED.

Typically, the reduction step is carried out by reacting compound of formula (V) with ketoreductase enzyme in the presence of cofactor for the ketoreductase and optionally a cofactor generating system. Ketoreductase enzymes are commercially available, for example, from Codexis, Inc.

The KRED can be found in a wide range of bacteria and yeast (for reviews: Kraus and Waldman, Enzyme catalysis in organic synthesis, Vols. 1 and 2.VCH Weinheim 1995; Faber, K., Biotransformations in organic chemistry, 4th Ed. Springer, Berlin Heidelberg New York. 2000; Hummel and Kula, 1989, Eur. J. Biochem. 184: 1-13). Several KRED gene and enzyme sequences have been reported, e.g., Candida magnoliae (Genbank Ace. No. JC7338; GL 1 1360538)Candida parapsilosis (Genbank Ace. No. BAA24528.1; GI:2815409), Sporobolomyces salmonicolor (Genbank Ace. No. AF160799; GL6539734).

The KRED can be a wild type or a variant enzyme. Sequences of wild type and variant KRED enzymes are provided in WO2005/017135, incorporated herein by reference. KRED enzymes are commercially available. Examples of these include but are not limited to KRED-101, KRED-119, KRED-130, KRED-NADH-101, KRED-NADH-110, KRED-P1-A04, KRED-P1-B02, KRED-P1-B05, KRED-P1-B05, KRED-P1-B10, KRED-P1-B12, KRED-P1-001, KRED-P1-H08, KRED-P1-H10, KRED-P2-B02, KRED-P2-0O2, KRED-P2-C11, KRED-P2-D03, KRED-P2-D11, KRED-P2-D12, KRED-P2-G03, KRED-P2-H07, KRED-P3-B03, KRED-P3-G09, KRED-P3-H12 and combinations thereof. Most preferably the enzyme is selected from the group consisting of KRED-P1-B12, KRED-P1-001, KRED-P1-H08, KRED-P1-H10 and combinations thereof.

Preferably, the ketoreductase is isolated. The ketoreductase can be separated from any host, such as mammals, filamentous fungi, yeasts, and bacteria. The isolation, purification, and characterization of a NADH-dependent ketoreductase is described in, for example, in Kosjek et al., Purification and Characterization of a Chemotolerant Alcohol Dehydrogenase Applicable to Coupled Redox Reactions, Biotechnology and Bioengineering, 86:55-62 (2004). Preferably, the ketoreductase is synthesized. The ketoreductase can be synthesized chemically or using recombinant means. The chemical and recombinant production of ketoreductases is described in, for example, in European Patent No. 0918090B. Preferably, the ketoreductase is synthesized using recombinant means in Escherichia coli. Preferably, the ketoreductase is purified, preferably with a purity of about 90% or more, more preferably with a purity of about 95% or more. Preferably, the ketoreductase is substantially cell-free.

As used herein, the term “cofactor” refers to a non-protein compound that operates in combination with a ketoredutase enzyme. Cofactors suitable for use with ketoreductase enzymes include, but are not limited to nicotinamide adenine dinucleotide phosphate (NADP⁺), reduced nicotinamide adenine dinucleotide phosphate (NADPH), nicotinamide adenine dinucleotide (NAD⁺) and reduced nicotinamide adenine dinucleotide (NADH). Generally the reduced form of the cofactor is added to the reaction mixture.

A cofactor regenerating system reduces the oxidized form of the cofactor. Cofactors oxidized by the kedoreductase-catalyzed reduction of the keto substrate are regenerated in reduced form by the cofactor regeneration system. The cofactor regenerating system may further comprise a catalyst, for example an enzyme catalyst.

Cofactor regeneration systems suitable for use with ketoreductase enzymes include, but are not limited to glucose and glucose dehydrogenase (GDH), formate and formate dehydrogenase, glucose-6-phosphate and glucose-6-phosphate dehydrogenase, a secondary alcohol and secondary alcohol dehydrogenase, phosphate and phosphate dehydrogenase, molecular hydrogen and hydrogenase, and the like.

Chemical cofactor regeneration systems comprising a metal catalyst and a reducing agent, for example molecular hydrogen or formate, may also be used in combination with either NADP⁺/NADPH or NAD⁺/NADH as the cofactor.

As used herein, the term “formate” refers to formate anion (HCOO⁻), formic acid (HCOOH) and mixtures thereof. Formate may be in the form of a salt, typically an alkali or ammonium salt (for example, HCOONa, KHCOONH4, NH₄HCO₂, and the like), in the form of formic acid, or mixtures thereof.

Suitable secondary alcohols include lower secondary alcohols and aryl-alkyl carbinols. Examples of lower secondary alcohols include isopropanol, 2-butanol, 3-methyl-2-butanol, 2-pentanol, 3-pentanol, 3,3-dimethyl-2-butanol, and the like. In a particularly preferred embodiment the secondary alcohol is isopropyl alcohol (IPA). Suitable aryl-akyl carbinols include unsubstituted and substituted 1-arylethanols.

The reduction step is preferably carried out in a co-solvent. The co-solvent assists in enhancing solubility of compounds having poor water solubility, thereby increasing overall rate of the reaction. Suitable co-solvents include organic solvents, for example methanol, IPA,1-octanol, ethyl acetate, methyl acetate, butyl acetate, heptane, octane, methyl t-butyl ether(MTBE), dimethyl sulfoxide (DMSO), tetrahydrofuran (THF), 2-methyltertahydrofuran, toluene and the like (including mixtures thereof), and ionic liquids, for example 1-ethyl 4-methylimidazolium tetra fluoroborate, 1-butyl-3-methylimidazolium tetrafluoroborate, 1-butyl-3-methylimidazolium hexafluoro phosphate, and the like. In some embodiments, aqueous solvents, including water and aqueous co-solvent systems, may be used. In a particularly preferred embodiment, DMSO is used as a co solvent.

The ratio of water to organic solvent in the co-solvent system is typically .in the range of from about 90:10 to about 95:05 (v/v) water to organic solvent. Preferably the solvent does not exceed 5% of the total volume of the reaction solution. The co-solvent system may be pre-formed prior to addition to the reaction mixture, or it may be formed in situ in the reaction vessel.

The aqueous solvent (water or aqueous co-solvent system) may be pH-buffered or unbuffered. Generally, the reduction can be carried out at a pH of about 10 or below, usually in the range of from about 5 to about 10. In some embodiments, the reduction is carried out at a pH of about 9 or below, usually in the range of from about 5 to about 9. In some embodiments, the reduction is carried out at a pH of about 8 or below, often in the range of from about 5 to about 8, and usually in the range of from about 6 to about 8. The reduction may also be carried out at a pH of about 7.8 or below or 7.5 or below. In a preferred embodiment, the reduction is carried out at neutral pH, i.e., about 7.

During the course of the reduction reactions, the pH of the reaction mixture may change. The pH of the reaction mixture may be maintained at a desired pH of 7 or within a desired pH range by the addition of an acid or a base during the course of the reaction. Alternatively, the pH may be controlled by using an aqueous solvent that comprises a buffer. Suitable buffers to maintain desired pH ranges are known in the art and include, for example, phosphate buffer, triethanolamine buffer, and the like. Combinations of buffering and acid or base addition may also be used.

Suitable bases for neutralization are organic bases, for example amines, alkoxides and the like, and inorganic bases; for example, hydroxide salts (e.g., NaOH), bicarbonate salts (e.g. NaHCO₃), icarbonate salts (e.g. K₂CO₃), basic phosphate salts (e.g. K₂HPO₄, Na₃PO₄), and the like.

Suitable acids to add during the course of the reaction to maintain the pH include organic acids, for example carboxylic acids, sulfonic acids, phosphonic acids, and the like, mineral acids, for example hydrohalic acids (such as hydrochloric acid), sulfuric acid, phosphoric acid, and the like, acidic salts, for example dihydrogenphosphate salts (e.g., KH₂PO₄), bisulfate salts (e.g., NaHSO₄) and the like. Some embodiments utilize formic acid, whereby both the formate concentration and the pH of the solution are maintained.

The reduction step is typically carried out at a temperature in the range of from about −70° C. to about 75° C. Preferably, the reduction step is carried out at a temperature in the range of from about −10° C. to about 55° C. In still other embodiments, it is carried out at a temperature in the range of from about 20° C. to about 45° C. In a particularly preferred embodiment the reaction is carried out under ambient conditions.

The ketoreductase are capable of reducing the ketone to alcohol with a stereomeric excess at least about 99% and is capable of converting at least about 90% of the ketone to alcohol.

The invention provides the compound of formula (IV) having an enantiomeric purity greater than about 95%, and, more preferably, greater than about 99%, as determined by HPLC.

Further, the enzymatic reduction process is environmently advantageous as compared to the prior art process wherein chiral amine are used in the prior art. The use of an enzyme as the reducing agent is cheaper compared to the use of a chiral amine. In addition, resolution using chiral amine according to known methods leads to about 50% loss of undesired isomer and hence it is not industrially suitable.

The compound of formula (IV) obtained by the process of the present invention is further condensed with compound of formula (III) to obtain compound of formula (II).

The condensation reaction is carried out in the presence of a suitable base in the presence of a suitable solvent. Suitably, the base comprises one or more of inorganic bases comprising alkali metal hydroxide, alkali metal carbonates, alkoxides or organic bases comprising primary, secondary, tertiary and heterocyclic amines and the suitable solvent comprises one or more of polar protic or aprotic solvent. The condensation step may be carried out at a temperature range of 30° C. to the boiling temperature of the solvent.

In another embodiment of the invention, the compound of formula (II) is hydrolyzed to obtain an endothelin receptor antagonist of formula (I).

In oneembodiment, the hydrolysis is carried out in the presehce of suitable base in a suitable solvent. Suitably, the base comprises one or more inorganic bases, such as an alkali metal hydroxide (for example, LiOH, NaOH and/or KOH), an alkali metal carbonate (for example, Li₂CO₃, Na₂CO₃ and/or K₂CO₃), or a mixture thereof. Examples of suitable solvents include polar solvents such as water, alcohols such as methanol and ethanol; ethers such as THF, 1,4-dioxane, diiospropyl ether, dibutyl ether and MTBE; esters comprising ethyl acetate, methyl acetate and propyl acetate; and mixture thereof.

The hydrolysis is typically carried out at a temperature in the range of from about −30° C. to about 50° C. Preferably, the hydrolysis is carried out at a temperature in the range of from about 35° C. to about 45° C. In still other embodiments, it is carried out at a temperature in the range of from about 40° C. to about 45° C.

The hydrolysis process of the present invention is advantageous as the process for preparing compound (I) does not involve high temperature and therefore is suitable industrially. By contrast, in WO2010/070658, in example 13, the hydrolysis is carried out at 80-90° C. This has disadvantages as it forms impure compound (I) with total impurities about 5% and chiral purity about 93-95% which requires repetitive purifications.

Another advantage of the low temperature hydrolysis process of the present invention is that the compound (I) is obtained with purity at about 99.8% and chiral purity 99.45%.

The compound of formula (I), may be optionally purified in the suitable solvent selected from an alcohol such as methanol, ethanol, isopropanol, butanol; N-methylpyrrolidone (NMP), DMSO, N,N-dimethylformamide (DMF), THF, water, andmixtures thereof.

Compounds (I), (II) and (IV) as shown above are depicted in the form of the (S)-isomer and are substantially free from (R)-isomer.

The endothelin receptor antagonists of formula (I) are substantially free from (R)-isomer.

According to another aspect of the present invention, there is provided a process for preparing a compound of formula (V) as exemplified in Scheme 2.

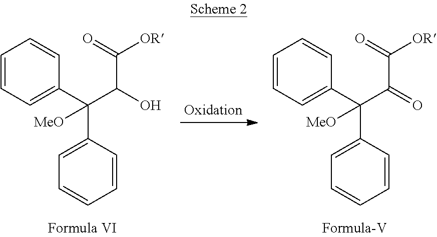

wherein R′ is an lower alkyl group which may be a straight or branched C₁-C₆ alkyl. group, such as methyl, ethyl, propyl, butyl, pentyl or hexyl.

In one embodiment, a compound of formula (VI) is oxidized to a compound of formula (V) using Dess Martin Periodinane (DMP) in the presence of an inert solvent. Suitably, the oxidation is performed in anon-polar solvent such as dichloromethane or chloroform, or a mixture thereof. The reaction is preferably performed at a temperature ranging from about 20 to about 30° C. The reaction usually completes within 0.5-2 hours. The compound of formula (V) may be conveniently separated from the carbonyl compound iodinane and acetic acid byproducts after basic work-up. The oxidation reaction using DMP includes milder conditions, shorter reaction times, higher yields, and simplified workups and is therefore advantageous.

In an alternative embodiment, a compound of formula (VI) may be oxidized to a compound of formula (V) by Swern oxidation. The oxidation preferably involves reaction of a compound of formula (VI) with DMSO, a dehydration agent such as oxalyl chloride or trifluoroacetic anhydride, and an organic base, such as triethylamine or diisopropylethylamine. The oxidation is carried out in the presence of a suitable solvent, such as dichloromethane, ethyl acetate or mixture thereof. The reaction temperature is preferably in the range from about −70 to about −50° C., more preferably from about −60 to about −55° C.

Compounds of formula (VI) may be prepared by known methods, for example by condensation of benzophenone with methyl-2-chloroacetate. This reaction is disclosed in U.S. Pat. No. 5,932,730 and may be carried out in accordance with the process disclosed therein.

In one embodiment, the present invention provides an enantiomerically pure compound of formula (IV), that is substantially pure of other process related impurities and optical impurities. The present invention further provides a process for preparing a compound of formula (IV) from compound of formula (V), which process advantageously does not require any purification, by techniques like chiral chromatographic separation or salt formation or recrystallization.

The present invention further, provides a process for preparing a compound of formula (V) by oxidation of a compound of formula (VI), wherein, advantageously the chemical purity is retained in the compound of formula (V) without performing any additional step of recrystallization or purification(s).

According to yet another aspect of the present invention, there is provided an alternate process for preparing a compound of formula (IV) as exemplified in Scheme 3 below.

Accordingly, in an embodiment, the invention provides an improved process for the preparation of compound of the formula (IV); wherein a compound of formula (VII) is resolved using a suitable optically active chiral amine base, such as (S)-(−)-1-(1-Naphthyl)ethyl amine or S-1-(4-nitrophenyl)ethyl amine, at a suitable low temperature ranging from about 25 to about 30° C. in a polar solvent to obtain a (S)-(+1-(1-Naphthyl)ethyl amine salt or S-1-(4-nitrophenyl)ethyl amine salt of compound of formula (VIII). Thereafter, the compound of formula (III) may be converted to a base, followed by esterification with a suitable esterifying agent such as dimethyl sulphate, in the presence of a suitable base such as sodium methoxide to obtain a compound of formula (IV).

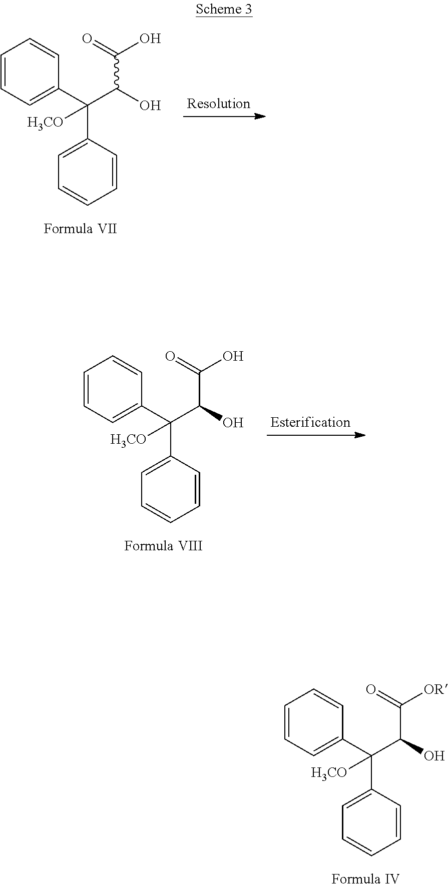

wherein, wherein R′ is an lower alkyl group which may be a straight or branched C₁-C₆ alkyl group, such as methyl, ethyl, propyl, butyl, pentyl or hexyl.

In one embodiment, a compound of formula (IV) is condensed with compound of formula (III) as hereinbefore described to obtain compound of formula (II), which is hydrolyzed and optionally further purified by crystallization from one or more solvents to obtain pharmaceutically acceptable grade endothelin receptor antagonist of formula (I).

In one embodiment, the present invention provides a substantially pure endothelin receptor antagonist of formula (I). As used herein, substantially pure refers to chemical and optical purity of greater than about 97%, preferably greater than about 98%, and more greater than about preferably 99.0% by weight.

The following non-limiting Examples illustrate the processes of the present invention.

### EXAMPLE 1

### Preparation of methyl-3-methoxy-2-oxo-3,3-diphenylpropionate (compound V; R′=methyl) (Dess Martin Periodinane Oxidation)

To a stirred solution of methyl-2-hydroxy-3-methoxy-3,3-diphenylpropionate (compound VI, R′=methyl) (290 gms/1.01 moles) in dichloromethane (5.8 lit) was charged Dess Martin Periodinane (643 gms/1.51 moles) under a dry flask under nitrogen at 20-25° C. The reaction mass was stirred for 1 hour and quenched in a cooled solution of 25% sodium thiosulfate and 25% sodium bicarbonate (1:1/5 lit) maintaining temperature below 20° C. The organic phase was washed with 2% sodium bicarbonate solution followed by water. The organic phase was dried on sodium sulphate, distilled off completely under vacuum and stirred in n-heptane (1.2 lit). The solid was isolated by filtration and dried to obtain 272.0 gms of the title compound.

Efficiency: 94.4

Purity by HPLC: 99.11%

### EXAMPLE 2

### Preparation of methyl-3-methoxy-2-oxo-3,3-diphenylpropionate (Compound V; R′=methyl) (Swern Oxidation)

To a stirred solution of oxalyl chloride (257.5 gms/2.02 moles) in dichloromethane (2.9 lit) was added DMSO (290 ml) maintaining temperature at -55 to −60° C. under nitrogen. To the reaction mass was added as solution of methyl-2-hydroxy-3-methoxy-3,3-diphenylpropionate (compound VI;R′=methyl) (290 gms/1.01 moles) in DMSO 2.9 lit) and stirred further for 20 minutes. Triethyl amine (1.45 lit) was added and the reaction mass was further stirred by maintaining temperature at −55 to −60° C. under nitrogen. Water (2.5 lit) was added and the phases were separated. Aqueous phase was extracted with dichloromethane (2.9 lit). The organic phases were combined together, washed with 10.0% HCl solution (5.8 lit) followed by water. The organic phase was dried on sodium sulphate, distilled off completely under vacuum and stirred in n-heptane (1.2 lit). The solid was isolated by filtration, and dried to obtain 285 gms of the title compound.

Efficiency: 98.9%

Purity by HPLC: 98.1%

### EXAMPLE 3

### Enzymatic Resolution

### Preparation of (S)-methyl-2-hydroxy-3-methoxy-3,3-diphenylpropionate (Compound IV; R′=methyl)

Preparation of Solution A: A mixture of potassium phosphate (2.32 gm, 13.36 mM), magnesium sulfate (0.033 gm, 0.13 mM), and NADP+(0.08 gm, 0.10 mM), was dissolved in 83.6 ml de-ionized water to obtain a clear solution.

Preparation of Solution B: In another flask was prepared solution of methyl-3-methoxy-2-oxo-3,3-diphenylpropionate [compound (V); 1.35 g/4.75 Mm] in 19 ml Isopropanol and 5 ml dimethyl sulphoxide.

The two solutions A & B were mixed together at 25-30° C. and agitated for 5 minutes. To the obtained solution, keto-reductase enzyme KRED-P1-B12 (0.095 g) was added and agitated at 25-30° C. for several hours. The progress of the reaction was monitored on HPLC till complete reduction of substrate to the corresponding alcohol was observed. After completion of reaction, the reaction mass was quenched by the addition of ethyl acetate (1 ml). The reaction mixture was filtered through sintered glass funnel to remove insoluble material. The filtrate was extracted with ethyl acetate (10 ml×3). The combined ethyl acetate extracts were concentrated under vacuum to give 1.25 g of the title compound (IV) as a white solid.

Efficiency: 92.6%

Enatiomeric excess >99%

Purity by HPLC: >97%

### EXAMPLE 4

### Preparation of (S)-methyl-2-(4,6-dimethylpyrimidin-2-yloxy)-3-methoxy-3,3-diphenylpropionate (Compound R′=methyl, R=methyl)

To a stirred solution of (S)-methyl-2-hydroxy-3-methoxy-3,3-diphenylpropionate (compound IV; 79 gms/0.276 moles) and potassium carbonate(41.9 gms/0.303 moles) in DMF (790 ml) was added 4,6-Dimethyl-2-(methyl sulfonyl)pyrimidine (compound III; R=methyl) (56.5 gms/0.303 moles) in a dry flask under nitrogen at 25-30° C. The reaction mass was heated to 90-92° C. and further stirred for 1 hour. The reaction mass was charged with ethyl acetate (2.4 lit). The organic layer was washed with 2N citric acid (500 ml) followed by water and treated with charcoal. The clear filtrate was distilled completely under vacuum. The residue was stirred with n-heptane (300 ml), filtered and dried to obtain 90 gms of compound (II).

The compound (II) was dissolved in acetonitrile (1.8 lit) and isolated in water (4.5 lit). The solid was isolated by filtration and dried to obtain 79 gms of compound (II).

Efficiency: 72.96%

Purity by HPLC:99.8%

Chiral purity: 99.97%

### EXAMPLE 5

### Preparation of (S)-2-(4,6-dimethylpyrimidin-2-yloxy)-3-methoxy-3,3-diphenyl propionic Acid (Compound I; R=methyl)

To a stirred solution of (S)-methyl-2-(4,6-dimethylpyrimidin-2-yloxy)-3-methoxy-3,3-diphenylpropionate (compound II; R′=methyl, R=methyl) (79 gms/0.201 moles) in 1,4-dioxane (790 ml) was added 2 N NaOH solution (18.6 gms of NaOH in 450.0 ml of DI Water). The reaction mass was heated to 40-45° C. and further stirred for 33 hours. The reaction mass was cooled to 25-30° C. and quenched in water (7.9 Lit). The reaction mass was cooled to 10-15° C. and acidified with conc. HCl. The solid was isolated by filtration and dried to yield 72.0 gms of the title compound (I).

Compound (I) was further purified in N-methyl pyrrolidone/n-heptane, followed by DMSO/n-heptane to obtain 65.0 gms of the title compound (I).

Efficiency: 85.33%

Chiral purity: 99.2%

Purity by HPLC: 99.8%

### EXAMPLE 6

### Preparation of (S)-methyl-2-(4,6-dimethoxypyrimidin-2-yloxy)-3-methoxy-3,3-diphenylpropionate (Compound II; R′=ethyl, R=methoxy)

To a stirred solution of (S)-methyl-2-hydroxy-3-methoxy-3,3-diphenylpropionate (compound IV;50 gms/0.174 moles) and potassium carbonate (26.36 gms/0.191 moles) in DMF (500 ml) was added 4,6-Dimethoxy-2-(methyl sulfonyl)pyrimidine (compound III; R=methoxy) (41.42 gms/0.191 moles) in a dry flask under nitrogen at 25-30° C. The reaction mass was heated to 90-92° C. and further stirred for 1.5 hour. The reaction mass was charged with ethyl acetate (1.5 lit).The organic layer was washed with 2N citric acid (500 ml) followed by water and treated with charcoal. The clear filtrate was distilled completely under vacuum. The residue was stirred with n-heptane (200 ml), filtered, washed with n-heptane (120 ml) and dried to obtain 56 gms of compound (II).

The compound (II) was dissolved in acetonitrile (1.12 lit) and isolated in water (2.8 lit). The solid was isolated by filtration and dried to obtain 49 gms of compound (II).

Efficiency: 66.10%

Purity by HPLC:99.1%

Chiral purity: 99.4%

### EXAMPLE 7

### Preparation of (.S)-2-(4,6-dimethoxypyrimidin-2-yloxy)-3-methoxy-3,3-diphenyl propionic acid (Compound I; R=methoxy)

To a stirred solution of (S)-methyl-2-(4,6-dimethoxypyrimidin-2-yloxy)-3-methoxy-3,3-diphenylpropionate (compound II; 49 gms/0.115 moles) in 1,4-dioxane (490 ml) was added 2 N NaOH solutions (10.6 gms of NaOH in 265.0 ml of distilled water). The reaction mass was heated to 40-45° C. and further stirred for 30 hours. The reaction mass was cooled to 25-30° C. and quenched in water (4.9 Lit). The reaction mass was cooled to 10-15° C. and acidified with conc. HCl. The solid was isolated by filtration and dried to yield 46.0 gms of the title compound (I).

Compound (I) was further purified in N-methyl pyrrolidone/n-heptane, followed by DMSO/n-heptane to obtain 41.0 gms of the title compound (I).

Efficiency: 86.53

Chiral purity:-99.1%

Purity by HPLC: 99.5%

### EXAMPLE 8

### Preparation of (S)-2-hydroxy-3-methoxy-3,3-diphenylpropionic acid (Compound VIII)

### (a) Resolution using (S)-(−)-1-(1-Naphthyl)ethyl amine

To a stirred solution of 2-hydroxy-3-methoxy-3,3-diphenylpropionic acid (compound VII; 240 gms/0.88 moles) in a mixture of tert-butyl methyl ether (1.2 lit) and methanol (1.2 lit) was added (S)-(−)-1-(1-Naphthyl) ethyl amine (82.8 gms/0.484 moles). The reaction mass was further stirred for 1 hour at 25-30° C. The solid was isolated by filtration, washed with tert-butyl methyl ether (500 ml) and dried. The solid was stirred in a mixture of distilled water (1.2 lit) and tert-butyl methyl ether (1.2 lit) and cooled to 10-15° C. The reaction mass was acidified with conc. HCl and stirred for 30 minutes. The organic phase was separated; aqueous phase was extracted with tert-butyl methyl ether (1.0 lit). The organic phases were combined together, washed with brine, and concentrated under vacuum at 25-30° C. The residue was stirred in n-Hepatne (720 ml). The solid was isolated by filtration and dried to give 79 g of the title compound (VIII).

Efficiency: 32.91%

Purity by HPLC: 99.5%

Chiral purity: 98.1%

### (b) Resolution using (S)-1-(4-nitrophenyl)ethyl amine

To a stirred solution of 2-hydroxy-3-methoxy-3,3-diphenylpropionic acid (compound VII; 240 gms/0.88 moles) in tert-butyl methyl ether (1.2 lit) was added S-1-(4-nitro phenyl)ethyl amine (73.5 gms/0.44 moles) at 45-50° C. The reaction mass was further stirred for 1 hour at 45-50° C., cooled to 25-30° C. and stirred for 1 hour. The solid was isolated by filtration, washed with tert-butyl methyl ether (500 ml) and dried. The solid was stirred in a mixture of distilled water (1.2 lit) and tert-butyl methyl ether (1.2 lit) and cooled to 10-15° C. The reaction mass was acidified with conc. HCl and stirred for 30 minutes. The organic phase was separated; aqueous phase was extracted with tert-butyl methyl ether (1.0 lit). The organic phases were combined together, washed with brine, dried on sodium sulphate and concentrated under vacuum at 25-30° C. The residue was stirred in n-Heptane (720 ml). The solid was isolated by filtration, and dried to give 66 g of the title compound (VIII).

Efficiency: 27.5%

Purity by HPLC: 99.78%

Chiral purity: 99.9%

### EXAMPLE 9

### Preparation of (S)-methyl-2-hydroxy-3-methoxy-3,3-diphenylpropionate (compound IV from compound VIII; R′=methyl)

Stirred (S)-2-hydroxy-3-methoxy-3,3-diphenylpropionic acid (compound VIII; 79 gms/0.290 moles) in dimethyl formamide (790 ml) under nitrogen and cooled to 5° C. Sodium methoxide (17.3 gms/0.320 moles) was added followed by slow addition of dimethyl sulfate (54.81 gms/0.44 moles) under nitrogen. The reaction mass was further stirred for 3 hours at 25-28° C. and quenched in DI water (2.4 lit). The reaction mass was extracted with ethyl acetate (3×790.0 ml), washed with water and distilled completely under vacuum below 50° C. to obtain 79 gms of the title compound (IV).

Efficiency: 95.11%

1. A process for preparing compound of formula (I), 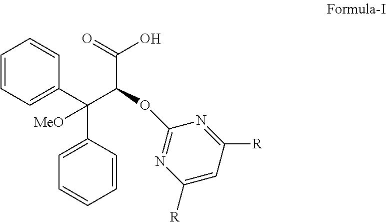
comprising converting a compound of formula (V) 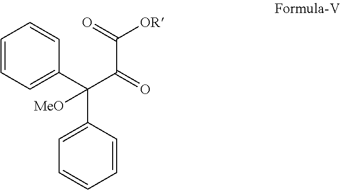
to compound of formula (I), wherein R is a methyl or a methoxy group and R′ in formula (V) is a lower alkyl group.

2. A process according to claim 1, wherein R is a methyl group.

3. A process according to claim 1 or claim 2, comprising reducing a compound of formula (V) with a chiral reducing agent to provide compound of formula (IV) 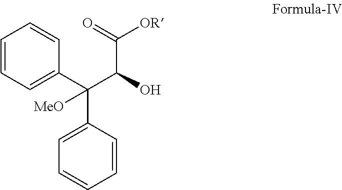
wherein, R′ is as defined in claim 1.

4. A process according to claim 3, wherein the chiral reducing agent is an enzyme, preferably a ketoreductase (KRED) or a carbonyl reductase.

5. A process according to claim 4, wherein the enzyme is selected from the group consisting of KRED-101, KRED-119, KRED-130, KRED-NADH-101, KRED-NADH-110, KRED-P1-A04, KRED-P1-B02, KRED-P1-B05, KRED-P1-B05, KRED-P1-B 10, KRED-P1-B 12, KRED-P1-001, KRED-P1-H08, KRED-P1-H10, KRED-P2-B02, KRED-P2-C02, KRED-P2-C11, KRED-P2-D03, KRED-P2-D11, KRED-P2-D12, KRED-P2-G03, KRED-P2-H07, KRED-P3-B03, KRED-P3-G09, KRED-P3-H12, and combinations thereof.

6. A process according to claim 5, wherein the enzyme is selected from the group consisting of KRED-P1-B12, KRED-P1-C01, KRED-P1-H08, KRED-P1-H10 and combinations thereof.

7. A process according to claim 5, wherein the reduction is carried in the presence of a cofactor for the ketoreductase and optionally a cofactor regenerating system.

8. A process according to claim 7, wherein the cofactor is selected from adenine dinucleotide phosphate (NADP⁺), reduced nicotinamide adenine dinucleotide phosphate (NADPH), nicotinamide adenine dinucleotide (NAD⁺) and reduced nicotinamide adenine dinucleotide (NADH).

9. A process according to claim 7, wherein the cofactor regenerating system comprises glucose and glucose dehydrogenase (GDH), formate and formate dehydrogenase, glucose-6-phosphate and glucose-6-phosphate dehydrogenase, a secondary alcohol (for example isopropanol) and secondary alcohol dehydrogenase, phosphate and phosphate dehydrogenase, or molecular hydrogen and hydrogenase.

10. A process according to claim 3, wherein the chiral reduction is carried in the presence of a cosolvent, preferably anorganic solvent.

11. A process according to claim 10 wherein the co-solvent is selected from the group consisting of methanol, isopropyl alcohol, 1-octanol, ethyl acetate, methyl acetate, butyl acetate, heptane, octane, methyl t-butyl ether, dimethyl sulfoxide, tetrahydrofuran, 2-methyltertahydrofuran, toluene and mixtures thereof.

12. A process according to claim 10 wherein the co-solvent is an ionic liquid.

13. A process according to claim 12 where in the ionic liquid is selected from the group consisting of 1-ethyl 4-methylimidazolium tetra fluoroborate, 1-butyl-3-methylimidazolium tetrafluoroborate, and 1-butyl-3-methylimidazolium hexafluoro phosphate.

14. A process according to claim 10 wherein the co-solvent is an aqueous solvent.

15. A process according to claim 14 wherein the aqueous solvent is selected from the group consisting of water and an aqueous co-solvent system.

16. A process according to claim 10, wherein the co-solvent is dimethyl sulfoxide.

17. A process according to claim 3, wherein the chiral reduction is carried out at a pH of about 10 or below, preferably at a pH of about 8 or below, more preferably at a pH of about 7.5 or below.

18. A process according to claim 17, wherein the pH of the reduction reaction is maintained by using an aqueous solvent that comprises a buffer.

19. A process according to claim 18, wherein the buffer comprises a phosphate buffer or a triethanolamine buffer.

20. A process according to claim 3, wherein the compound of formula (IV), has an enantiomeric purity of greater than about 99% as determined by HPLC.

21. A process according to claim 3, further comprising condensing a compound of formula (IV) with a 4,6-disubstituted-2-methyl sulfonyl pyrimidine of formula (III) 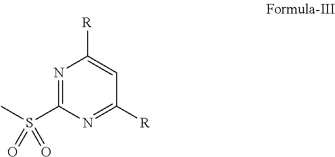
in the presence of a base in a suitable solvent to yield acompound of formula (II). 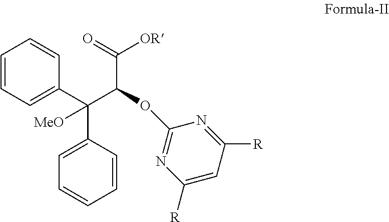
wherein, R and R′ are as defined in claim 1.

22. A process according to claim 21, wherein the base comprises one or more inorganic bases, preferably an alkali metal hydroxide, alkali metal carbonate, or alkali metal alkoxide.

23. A process according to claim 21, wherein the base comprises one or more organic bases, preferably a primary, secondary, tertiary or heterocyclic amine.

24. A process according to claim 21, wherein the solvent comprises one or more polar protic or aprotic solvents.

25. A process according to claim 21, wherein the condensation is carried out at a temperature in the range of from about 30° C. to the boiling temperature of the solvent.

26. A process according to claim 21, further comprising hydrolysing a compound of formula (II) in the presence of a suitable base, in a suitable solvent to obtain a compound of formula (I) 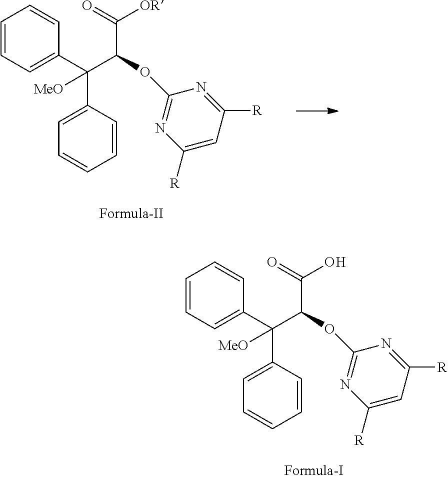
wherein, R and R′ are as defined in claim 1.

27. A process according to claim 26, wherein the base is an inorganic base selected from the group consisting of alkoxides, alkali metal hydroxide or alkali metal carbonates and/or an organic base selected from the group consisting of primary, secondary, tertiary or heterocyclic amines.

28. A process according to claim 26, wherein the solvent is a polar solvent, preferably selected from the group consisting ofwater, an alcohol, an ether, an ester, or a mixture thereof.

29. A process according to claim 28, wherein the ether is selected from the group consisting of THF, 1,4-dioxane, diiospropyl ether, dibutyl ether, MTBE or a mixture thereof; and wherein the ester is selected from the group consisting of ethyl acetate, methyl acetate, propyl acetate or a mixture thereof.

30. A process according to claim 26, wherein the compound of formula (I) is further purified by crystallisation from a solvent selected from the group consisting of an alcohol (such as methanol, ethanol, isopropanol, butanol or a mixture thereof); N-methylpyrrolidone, dimethyl sulfoxide, dimethyl formamide, tetrahydrofuran, water or a mixture thereof.

31. A compound of formula (V) 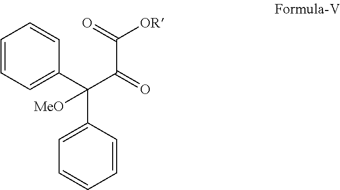
wherein R′ is an lower alkyl group which may be a straight or branched C₁-C₆ alkyl group.

32. A compound according to claim 31, wherein R′ is methyl, propyl, butyl, pentyl or hexyl, preferably methyl.

33. A process for preparing a compound of formula (V) according to claim 31. 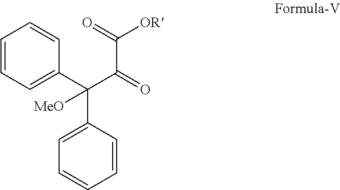
comprising: converting a compound of formula (VI) 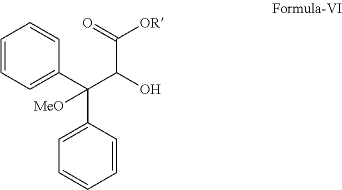
to acompound of formula (V), wherein R′ is as defined in claim 31.

34. A process according to claim 33, comprising oxidising a compound of formula (VI).

35. A process according to claim 34, wherein the oxidising agent is Dess Martin Periodinane (DMP).

36. A process according to claim 35, wherein the oxidation is carried out in a non-polar solvent selected fromdichloromethane or chloroform, preferably at a temperature in the range from about 20 to about 30° C.

37. A process according to claim 33, comprising converting a compound of formula (VI) to acompound of formula (V) by Swern oxidation.

38. A process according to claim 37, comprising reacting a compound (VI) with dimethyl sulfoxide, a suitable dehydration agent, and an organic base

39. A process according to claim 38, wherein the dehydration agent is selected from oxalyl chloride or trifluoroacetic anhydride.

40. A process according to claim 38, wherein the organic base is triethylamine or diisopropylethylamine.

41. A process according to claim 37, wherein the oxidation is carried out in the presence of a solvent selected from the group consisting of dichloromethane, ethyl acetate or a mixture thereof, preferably at a temperature in the range from about −70 to about −50° C., more preferably in the range of from about −60 to about −55° C.

42. A method for preparing an endothelin receptor antagonist, comprising using Use of a compound of formula (V) as claimed in claim 31 in the preparation of the endothelin receptor antagonist.

43. The method Use according to claim 42, wherein the endothelin receptor antagonist is a compound of formula (I).

44. A process according to claim 1, wherein the compounds of formula (I) , formula (II) and formula (IV) are substantially free from R-isomer.

45. (canceled)
